(19) United States
(12) Patent Application Publication
(10) Pub. No.: US 2003/0124591 A1
(43) Pub. Date: Jul. 3, 2003

(54) Notch 4 and schizophrenia

(76) Inventors: Jun Wei, Bangor (GB); Gwynneth Hemmings, Bangor (GB)

Correspondence Address: JACOBSON HOLMAN PLLC, 400 SEVENTH STREET N.W., SUITE 600, WASHINGTON, DC, 20004, US

(21) Appl. No.: 10/275,975

(22) PCT Filed: May 31, 2001
(86) PCT No.: PCT/GB01/02429

(30) Foreign Application Priority Data
May 31, 2000 (GB) 0013212.6

(51) Int. Cl. C12Q 1/68; A61K 39/395

(52) U.S. Cl. 435/6; 424/146.1

(57) ABSTRACT

Methods are provided of diagnosing, treating and testing a subject's susceptibility for schizophrenia, based on investigation of the Notch 4 gene or gene(s) associated therewith. Agents which modify the function of the Notch 4 gene are described used in the treatment of schizophrenia.

Schizophrenia is an illness which causes a great deal of distress to both patients and relatives. Although there are many drugs which modify the symptoms of schizophrenia, their effect sizes are small. On average, the drugs relieve only 15-25% of symptoms as measured by standard rating scales, so leaving 75-85% of symptoms untouched. The drugs have little effect on long term recovery from the illness: long term outcome has not changed over the past 100 years (Hegarty et al, Am J Psychiatry 1994: 151:1409-16). There is therefore a great need for the development of new treatment approaches.

For some time attempts have been made to identify the major genes involved in schizophrenia. This is a field where many genes and chromosome regions have been identified as possibly involved, only for the results to fail to be replicated by others. Several studies have suggested that there is in the vicinity of the human chromosome 6p a susceptibility locus for schizophrenia. [Wang, S. et al. Nature Genet. 10, 41-46 (1995); Straub, R. E. et al. Nature Genet. 11, 235-236 (1995); Schwab, S. G. et al. Nature Genet. 11, 325-327 (1995); Schizophrenia Linkage Collaborative Group (Schizophrenia Linkage Collaborative Group for Chromosomes 3, 6 and 8) Am. J. Med. Genet. 67, 580-694 (1996). No firm conclusions have been drawn yet. The 6p21 region has now been fully mapped and the complete genomic DNA sequence is in the public domain.

The NOTCH family of genes was first identified in Drosophila. These genes encode transmembrane receptor proteins with intracellular segments which are able to interact with signal transduction processes. The Notch genes are involved in differentiation and in cell-cell interactions. They have been implicated in the development of many tissues, including the nervous system. Notch 4 is a gene which has a specific role in regulating capillary and endothelial developments. The gene, and various ways of modulating its function and the functions of its gene products have been described in international patent application no PCT WO 98/57621. The practical applications described in this patent specification are related entirely to the modification of angiogenesis in either an upwards or downwards direction, depending on the nature of the condition to be treated. Modulation of Notch 4 signalling can be used to modulate angiogenesis either positively, by activating Notch signalling to stimulate angiogenesis or negatively, by blocking Notch signalling to block angiogenesis. This induction or inhibition of angiogenesis in vivo can be used as a therapeutic means to treat a variety of diseases including cancer, diabetes, wound repair and arteriosclerosis.

The present invention relates to the significance of Notch 4 protein with regard to schizophrenia and provides a method of diagnosing schizophrenia using the Notch 4 gene sequence, mRNA or proteins derived therefrom.

The present invention further provides in the treatment of schizophrenia use of agents which modify the function of the Notch 4 gene. For example, there may be provided a pharmaceutical composition for the treatment of schizophrenia comprising an amount of an antagonist agent effective to reduce the Notch 4 signalling. Alternatively, such a composition may comprise an agonist agent effective to activate Notch 4 signalling. These agents may be present with a pharmaceutically acceptable carrier.

A method of treatment of schizophrenia is provided comprising administering to the subject an effective amount of an agent which modulates Notch 4 signalling.

Antibodies effective to block binding of a ligand to the Notch 4 protein may also be used.

The present invention also provides agents which imitate the function of the Notch 4, gene at the level of the DNA, the mRNA, the protein or the receptors or binding sites for the protein. Such agents may be used in a method for treatment of schizophrenia.

Notch 4 is a member of a highly conserved group of genes which are consistently found together in many species. These are the genes for Notch 4 itself and the genes for the pre-B cell leukaemia transcription factor 2 (PBX-2), the extracellular matrix protein gene, tenascin-XA (TNX-A), the receptor for advanced glyosylation end products (RAGE) and the enzyme lysophosphatidic acid acyl transferase (LPAT). It is possible that the abnormality on Notch 4 could exert its effects by modifying the function of one of these four other genes with which Notch 4 is so consistently associated.

The present invention further provides genes which are associated with the Notch-4 gene and agents which act to modify the functions of such genes and their use in the treatment of schizophrenia.

Agents used in the present invention may be nucleotide or amino acid sequences, or proteins or antibodies which interact with proteins, or small molecules which imitate or antagonise the effects of the gene, the mRNA or the protein products of the gene.

The present invention provides a susceptibility test for schizophrenia comprising the step of comparing the sequence of the Notch 4 gene of the subject with the known sequence of a non-mutated Notch 4 gene of a healthy subject and discovering that there are mutation(s). These mutation(s) may be identified and attributed to specific mutations known to cause or give rise to susceptibility to schizophrenia.

Experimental Data

Access was obtained to a group of families with schizophrenic members. Using densely spaced DNA markers, the transmission disequilibrium test (TDT) was used to search for a susceptibility gene within the available human major histocompatibility complex (MHC) region mapped to 6p21.3. Eighty Caucasian parent-offspring trios, consisting of fathers, mothers and affected offspring with schizophrenia were tested. 48 unaffected offspring were also recruited to assess the effect of segregation distortion. A two-step mapping process was performed: the first to identify a region associated with schizophrenia; and the second to narrow down the region to see if a susceptibility gene for schizophrenia could be found. Thirteen loci of sequestered genomic DNA, containing highly polymorphic microsatellites were retrieved from the GenBank databases. The order of these 13 loci in the region of interest is as follows: 6pter-AC004810-HSY14768DJ201G24-HSA012008-HSMC3W36A-HSMHC3A5-HS1077I5-HS172K2-HSDV19-HSEVMHC-HS014-HS1033B10-HSICK721Q-6cen.

As shown in Table 1, the TDT analysis showed that the HSMHC3A5 locus was significantly associated with schizophrenia. This locus contains the Notch 4 gene, with a putative promoter region and 30 exons.

TABLE 1
Linkage disequilibrium analysis of the class II and
class III regions of the MHC in schizophrenia

| Locus | Accession | Marker^{a} | Heterozygosity | P-value^{b} |
|---|---|---|---|---|
| AC004180 | AC004180 | (GT)n | 0.85 | 0.145 |
| HSY14768 | Y14768 | (CA)n | 0.82 | 0.16 |
| DJ201G24 | AF129756 | (GT)n | 0.82 | 0.311 |
| HSA012008 | AJ012008 | (CA)n | 0.67 | 0.463 |
| HSMC3W36A | U89337 | (TTTC)n | 0.73 | 0.5 |
| HSMHC3A5 | U89335 | (TAA)n^{c} | 0.74 | 0.00017 |
| | | SNP1^{d} | 0.16 | 0.267 |
| | | SNP2^{e} | 0.26 | 0.002 |
| | | (CTG)n^{f} | 0.79 | 0.000036 |
| | | (TTAT)n^{g} | 0.51 | 0.393 |
| HS1077I5 | AL034394 | (CT)n | 0.66 | 0.356 |
| HS172K2 | Z84814 | (GT)n | 0.75 | 0.5 |
| HSDV19 | Z84490 | (CA)n | 0.73 | 0.372 |
| HSEVMHC | X87344 | (TAAA)n | 0.59 | 0.016 |
| HSO14 | Z84497 | (GT)n | 0.76 | 0.5 |
| HS1033B10 | AL031228 | (CCTT)n | 0.53 | 0.5 |
| HSICK721Q | AL021366 | (GT)n | 0.83 | 0.108 |
| notes: | | | | |
| ^{a}The microsatellites, including di-, tri- or tetra- nucleotide repeats, were genotyped using a PCR-based process. The PCR products were electrophoresed on a 12% polyacrilamide gel followed by ethidium bromide staining. To enhance the resolution of PCR fragments by gel electrophoresis, the primers were designed so that the predicted length of the PCR products was in the range of 90-130 bp according to the known sequence data retrieved from the GenBank databases. | | | | |
| # The SNP markers at the HSMHC3A5 locus were identified by DNA sequencing analysis. SNP1 is a T to C base change, which creates a MspI site, and SNP2 is an A to G base change, which also creates a MspI site. Both SNPs were genotyped by the PCR-based restriction fragment length polymorphism analysis. | | | | |

^{b}The likelihood-based linkage disequilibrium analysis program for the McNemar test (Terwilliger, J. D. Am. J. Hum. Genet. 56, 777-787 (1995)), the TDTLIKE program, was applied to analyse the genotyping data. The significance level of a P-value was set at 0.003, as 17 marker loci were tested totally in this study.
^{c}Eight alleles were typed due to the (TAA)n repeat, including (TAA)₇, (TAA)₈, (TAA)₉, (TAA)₁₀, (TAA)₁₁, (TAA)₁₂, (TAA)₁₃ and (TAA)₁₄. Frequencies of the individual alleles transmitted by 119 heterzygotic parents were 1, 53, 22, 22, 5, 7, 5 and 4, and those non-transmitted were 1, 21, 35, 32, 7, 13, 4 and 6.
^{d}Frequencies of the 2 individual alleles transmitted by 26 heterozygotic parents were 30 and 22.
^{e}Frequencies of the 2 individual alleles transmitted by 41 heterozygotic parents were 27 and 55.
^{f}Seven alleles were typed due to the (CTG)n repeat, including (CTG)₅, (CTG)₆, (CTG)₉, (CTG)₁₀, (CTG)₁₁, (CTG)₁₂ and (CTG)₁₃. Frequencies of the individual alleles transmitted by 126 heterozygotic parents were 1, 13, 23, 63, 8, 17 and 1, and those non-transmitted were 1, 18, 47, 23, 12, 22 and 3.
^{g}Six alleles were typed due to the (TTAT)n repeat, including (TTAT)₉, (TTAT)₁₀, (TTAT)₁₁, (TTAT)₁₂, (TTAT)₁₃ and (TTAT)₁₄. Frequencies of the individual alleles transmitted by 69 heterozygotic parents were 5, 0, 31, 20, 11 and 2, and those non-transmitted were 9, 3, 24, 23, 9 and 1. Since the (TTAT)n repeat is unstable (but not expandable) in some families, only 68 of 80 parent-offspring trios were available for the TDT anaylsis.

In order to narrow down the region further, a second association study was conducted using 3 microsatellites and 2 single nucleotide polymorphism (SNP) markers within the HSMHC3A5 locus (Table 1). These markers are a (TAA)n repeat, and SNP1 and SNP2 in the 5′-flanking region of the Notch 4 gene, the (CTG)n repeat in exon 1 and the (TTAT)n repeat in intron 17. The 48 unaffected offspring were typed using these five markers and compared with the schizophrenic offspring as shown in Table 2. The TDT did not show significant distortion at the five marker loci. The SNP-2 and (CTG)n regions showed very strong associations with schizophrenia suggesting that the disease-related mutation(s)may occur in or near the promoter and exon-1 regions. It is to be determined whether these represent loss of function or gain of function mutations.

TABLE 2
Haplotype analysis for association
of the Notch 4 gene variation with schizophrenia

| Haplotype | P-value |
|---|---|
| (TAA)n-SNP1 | 0.0002 |
| SNP1-SNP2 | 0.0162 |
| SNP2-(CTG)n | 0.0000078 |
| (CTG)n-(TTAT)n | 0.189 |
| (TAA)n-SNP1-SNP2 | 0.000028 |
| SNP1-SNP2-(CTG)n | 0.000011 |
| SNP2-(CTG)n-(TTAT)n | 0.064 |

The (TAA)n repeat is about 8.8 kb away from the SNP1 site; SNP1 is present at base −1725 of the 5′-flanking region of the Notch 4 gene, and SNP2 at base −25 within the promoter region; and the (CTG)n repeat is located at exon 1 of the gene, just 40 bp away from the SNP2 site and about 12.3 kb away from the (TTAT)n repeat.

The (CTG)n repeat is involved in coding for the leucine residue in the signal peptide domain of Notch 4. In this study, 7 individual alleles were totally typed due to the (CTG)n repeat in the Caucasian population, although only 4 alleles were observed in a Japanese population (Ando, A. et al Tiss. Antig. 50, 66-70 (1997)). An excess of the (CTG)₁₀ allele was transmitted to affected offspring by their parents. No expansion of the (CTG)n repeat was observed in affected individuals. Possibly the (CTG)₁₀ allele itself could contribute to the aetiology of schizophrenia. On the other hand, SNP2 of the A to G base change in the promoter region should also be considered as being an important candidate site. It is possible that the A-G substitution could affect the transcription of the Notch 4 gene. In any case, the results strongly support the possibility that the Notch 4 gene may confer a susceptibility to schizophrenia.

1. A method for diagnosing schizophrenia using the Notch 4 gene sequence, mRNA or proteins derived therefrom.

2. A method for the treatment of schizophrenia using a composition comprising an agent which modifies the function of the Notch 4 gene or gene(s) associated therewith, or the use of the said agent in the manufacture of a medicament for the treatment of schizophrenia.

3. The method or use of claim 2, wherein the agent is an antagonist agent effective to reduce the Notch 4 signalling.

4. The method or use of claim 2, wherein the agent is an agonist agent effective to activate Notch 4 signalling.

5. The method or use of claim 2, wherein the agent comprises antibodies effective to block binding of a ligand to the Notch 4 protein.

6. A pharmaceutical composition for the treatment of schizophrenia comprising an agent which modifies the function of the Notch 4 gene or a gene(s) associated therewith.

7. The pharmaceutical composition of claim 6, wherein the agent is an antagonist agent effective to reduce the Notch 4 signalling.

8. The pharmaceutical composition of claim 6, wherein the agent is an agonist agent effective to reduce the Notch 4 signalling.

9. The pharmaceutical composition of claim 6, wherein the agent comprises antibodies effective to block binding of a ligand to the Notch 4 protein.

10. A method for the treatment of schizophrenia using agent(s) which imitate the function of the Notch 4 gene at the level of the DNA, the mRNA, the protein or the receptors or binding sites for the protein, or the use of the said agent(s) in the manufacture of a medicament for the treatment of schizophrenia.

11. A method for testing a subject's susceptibility for schizophrenia comprising:
comparing the sequence of the Notch 4 gene of the subject with a known sequence of a non-mutated Notch 4 gene of a healthy subject and discovering that there are mutation(s);
identifying the said mutation(s); and
attributing the said specific mutation(s) to those known to cause or give rise to susceptibility to schizophrenia.
